(19) United States
(12) Patent Application Publication
(10) Pub. No.: US 2023/0361562 A1
(43) Pub. Date: Nov. 9, 2023

(54) PROTECTION FOR SINGLE FAULT IN WIRELESS POWER TRANSFER

(71) Applicant: Medtronic, Inc., Minneapolis, MN (US)

(72) Inventors: Andrew T. Fried, St. Paul, MN (US); Charles M. Nowell, JR., Longwood, FL (US); Brett A. Otteson, Minneapolis, MN (US); Venkat R. Gaddam, Plymouth, MN (US); Robert J. Monson, St. Paul, MN (US)

(21) Appl. No.: 18/309,176

(22) Filed: Apr. 28, 2023

Related U.S. Application Data
(60) Provisional application No. 63/364,101, filed on May 3, 2022.

(51) Int. Cl. H02J 3/00; H02J 50/10; H02J 50/20; G01R 22/06; A61M 5/142

(52) U.S. Cl. CPC H02J 3/00125; H02J 50/10; H02J 50/20; G01R 22/068; A61M 5/14276

(57) ABSTRACT

A wireless power transfer system and devices that are configured to perform techniques to detect a single fault in primary processing circuitry by using second, independent processing circuitry. The techniques may include calculating and verifying an integrated output power dose. Verifying the integrated output power dose may include, for example, secondary processing circuitry calculating the integral of power delivered over a predetermined time duration and compares the calculated integral to an expected integral dose curve stored at a memory location accessible by the secondary processing circuitry. The detection techniques may also include determining a maximum output power profile. The secondary processing circuitry may receive a commanded output power target from the primary processing circuitry and compare the commanded output power to the maximum allowed output power vs. time.

This application claims the benefit of U.S. Provisional Patent Application 63/364,101, filed 3 May 2022, the entire content of which is incorporated herein by reference.

### TECHNICAL FIELD

The disclosure relates medical devices, and specifically, transferring power to medical devices.

### BACKGROUND

Portable electronic devices may be located in locations where providing electrical power via a wired connection to a power source may be difficult or inconvenient to the end user. Some portable electronic devices may include a power supply such as a rechargeable battery or some other electrical energy storage device. In other examples, portable electronic devices may not include an internal power supply and instead be configured to directly receive wireless power to operate. Some examples of portable electronic devices may include electric vehicles, smart phones, smart watches, headphones, speakers, or computing devices. In other examples, portable electronic devices may include implantable medical devices or body worn devices. Implantable medical devices may receive wireless power via a transcutaneous power transfer configured to either directly power the device or to recharge an electrical energy storage device that provides power to the implantable medical device. Body worn devices such as an insulin pump may be recharged wirelessly with a charging pad for convenience to the user. Electric vehicles for personal use or for public transportation may be charged wirelessly for convenience to the user or to increase operating time or to improve autonomy if the vehicle is self-driving. The risks of overcharging the portable electronic device or overheating the device need to be managed according to the potential severity of failures that could occur such as rupturing the battery, creating shrapnel, creating a fire, or burning the user.

### SUMMARY

In general, the disclosure describes a wireless power transfer system and devices that are configured to perform techniques to detect a single fault in primary processing circuitry by using independent processing circuitry. The techniques may include calculating and verifying an integrated output power dose. Verifying the integrated output power dose may include, for example, secondary processing circuitry calculating the integral of power delivered over a predetermined time duration and compares the calculated integral to an expected integral dose curve stored at a memory location accessible by the secondary processing circuitry. The secondary processing circuitry may receive a commanded output power target from the primary processing circuitry and compare the commanded output power to the maximum allowed output power vs. time, e.g., to a maximum output power profile.

In one example, this disclosure describes a medical system that comprises a power transmitting unit with a power transmitting antenna; a driver circuit configured to cause the power transmitting antenna to output the wireless electrical energy; first processing circuitry operatively coupled to a first memory, the first processing circuitry configured to execute programming instructions stored at the first memory to determine and output a first target power magnitude; and second processing circuitry configured to: control the driver circuit; receive the first target power magnitude; independently calculate a second target power magnitude; compare the first target power magnitude to the independently calculated second target power magnitude; responsive to determining the first target power magnitude corresponds to the second calculated target power magnitude compare the first target power magnitude to a threshold output power limit; and responsive to determining that the first target power magnitude is outside the threshold output power limit, control the driver circuit to terminate power transmission.

In another example, this disclosure describes a power transmitting device comprising a power transmitting antenna; a driver circuit configured to cause the power transmitting antenna to transmit wireless electrical energy to an implantable medical device; first processing circuitry operatively coupled to a first memory, the first processing circuitry configured to execute programming instructions stored at the first memory to determine and output a first target power magnitude; and second processing circuitry configured to: control the driver circuit; receive the first target power magnitude; independently calculate a second target power magnitude; compare the first target power magnitude to the independently calculated second target power magnitude; responsive to determining the first target power magnitude matches the independently calculated second target power magnitude compare the first target power magnitude to a threshold output power limit; and responsive to determining that the first target power magnitude is outside the threshold output power limit, control the driver circuit to terminate power transmission.

In another example, this disclosure describes a method for delivering wireless power comprising sending, by first processing circuitry operatively coupled to a memory, a first target power magnitude to second processing circuitry, the second processing circuitry configured to control a driver circuit that is configured to drive a power transmitting antenna to transmit wireless electrical energy based on the first target power magnitude, wherein the wireless electrical energy provides wireless power to an implantable medical device; independently calculating, by the second processing circuitry, a second target power magnitude; comparing, by the second processing circuitry, the first target power magnitude to the independently calculated second target power magnitude; responsive to determining the first target power magnitude corresponds to the second target power magnitude comparing, by the second processing circuitry, the first target power magnitude to a threshold output power limit; responsive to determining that the first target power magnitude is outside the threshold output power limit, controlling, by the second processing circuitry, the driver circuit to terminate power transmission.

The details of one or more examples of the disclosure are set forth in the accompanying drawings and the description below. Other features, objects, and advantages of the disclosure will be apparent from the description and drawings, and from the claims.

### BRIEF DESCRIPTION OF DRAWINGS

FIG. 1 is a conceptual diagram illustrating an example system including a medical device and external computing devices according to one or more techniques of this disclosure.

FIG. 2 is a block diagram illustrating example components of the medical device of FIG. 1.

FIG. 3A is a block diagram of an example external computing device of FIG. 1.

FIG. 3B is a block diagram of an example external computing device of FIG. 1 that include a backup processor.

FIG. 4 is a graph illustrating example output power profile limits and integral dose curve limits according to one or more techniques of this disclosure.

FIG. 5 is a flowchart illustrating an example operation of the system of this disclosure.

FIG. 6 is a flowchart illustrating an example operation of the secondary processing circuitry of this disclosure.

### DETAILED DESCRIPTION

This disclosure describes devices, systems, and techniques relating to operating a wireless power transfer system controlled by primary processing circuitry and secondary, independent processing circuitry to detect a single fault in the operation of primary processing circuitry. Some systems may include only a single processor configured to control a power transfer system, such as receive information regarding power transfer characteristics and/or device charging status. However, if there is a fault with the single processor, the single processor may not appropriately calculate parameters for operating the power transfer system. Some examples of what may cause a single fault in the operation of the single processor, or circuitry, may include an issue with the single processor, a malfunction of a sensor, e.g., a voltage measurement error, a communication error as well as an issue caused by a first fault that the single processor may not detect.

The techniques of this disclosure may include calculating and verifying an integrated output power dose that may be used to control the wireless power transfer system. Verifying the integrated output power dose may include the secondary processing circuitry calculating the integral of power delivered over a predetermined time duration and compares the calculated integral to an expected integral dose curve stored at a memory location accessible by the secondary processing circuitry. By verifying the integrated power dose, the system may detect an output power magnitude that exceeds a threshold for an expected charging session duration. In addition, by verifying the integrated power dose, the system may also detect a correct output power magnitude but delivered for a longer than expected duration.

The detection techniques of this disclosure may also include comparing an output power to an output power threshold, or multiple output power thresholds over time in an output power profile. The output power threshold may also be referred to as a maximum allowable output power for a particular period of time of the charging session. The output power profile may include one or more output power thresholds associated with a span of time of a wireless power transfer session. The secondary processing circuitry may receive a commanded output power target from the primary processing circuitry at a first time and compare the commanded output power target to the output power threshold associated with the first time. In response to the commanded output power target value exceeding the output power threshold, e.g., greater than a limit associated with the first time, the secondary processing circuitry may declare a fault and reduce or cease delivering wireless power. In some examples, the output power profile may include a maximum duration based on the wireless power transfer mode. In this manner, comparison of the commanded output power to the output power threshold may detect other faults, such as an indefinite power transfer session duration, e.g., caused by a primary processing circuitry failure.

The techniques of this disclosure include several example implementations to independently verify the primary processing circuitry. In some examples, the secondary processing circuitry may be configured to control the “tank,” e.g., the primary coil and associated driver and compensation circuitry. The secondary processing may further be configured to receive a target power magnitude from the primary processing circuitry and control the driver circuitry to output wireless power at the target power magnitude via the primary coil. In a first example, the secondary processing circuitry is also configured to determine the threshold output power limit based on the time period of the charging session, compare the received target output power to the threshold output power limit and take some action, e.g., as described above, if the received target output power exceeds the threshold output power limit. For the first example, the secondary processing circuitry is configured to calculate and compare the integral power dose to the integral power dose limit, as described above.

In another example implementation, the secondary processing circuitry may be configured to independently receive at least some of the same input information used by the primary processing circuitry, then independently calculate the target power magnitude. The secondary processing circuitry is configured to compare the independently calculated target power magnitude to the target power magnitude received from the primary processing circuitry, along with the previously described comparison to the threshold output power limit and integral power dose limit. In some examples, the secondary processing circuitry may be a separate device from the primary processing circuitry. In other examples, the secondary processing circuitry may be a separate processing die located on the same device, e.g., on the same leadframe with the same encapsulant, as the primary processing circuitry. In other examples, the secondary processing circuitry may be implemented as another core of a single processor or where a single processor has multiple threads running in parallel.

In another example implementation, a third processing circuitry, e.g., a backup processing circuitry, is configured to independently receive at least some of the same input information used by the primary processing circuitry, then independently calculate the target power magnitude. The secondary processing circuitry, implemented as the backup processing circuitry, may receive the target power magnitude from both the primary processing circuitry and the backup processing circuitry. If both target power magnitudes match, e.g., within a threshold range of each other, the secondary processing circuitry may proceed to output the wireless power according to the target power magnitude. In some examples, the secondary processing circuitry may also perform the output power threshold comparison and integral power dose comparison. In other examples, the backup processing circuitry, or some combination of system processing circuitry, may perform the output power threshold comparison and integral power dose comparison.

If both target power magnitudes do not match, then the processing circuitry may take any one or more of several actions. One example action may be to flag an error and stop the wireless power transfer. Another example action may be to deliver wireless power at the lower of the two received target power magnitudes.

The detection techniques of this disclosure may provide advantages when compared with a single processor, e.g., the primary processor, controlling the wireless power transfer system without independent verification. A fault with the primary processor may cause the wireless power transfer system to output too much power or too little power. In some examples, outputting too much power may have undesirable effects, such as prematurely drain a battery of the power transmitting device, undesirable heating of the power receiving device (e.g., an implantable medical device), heating the tissue of the patient surrounding the medical device to a discomfort level, rupturing the power supply of the power receiving device, creating a fire due to overcharging the power supply of the power receiving device, and so on. Outputting too little power may cause a recharge session to take longer than needed, which may lead to user inconvenience (e.g., having to wait longer to recharge the wireless power receiver) ending a recharge session with less than fully recharged device, more frequent and longer charging sessions, and so on. These charging issues may result in patient dissatisfaction with the use of the medical device, which may reduce the efficacy of the intended therapy. In examples of other types of medical devices, charging issues may also result in user dissatisfaction. Note that when examples are given in terms of a medical device system and patient that these examples may also be applied to non-medical systems and users.

Tissue heating surrounding the medical device may increase tissue temperature and may depend on intensity and distribution of the electromagnetic field for recharging and the properties of the tissue as well as the materials of the medical device. In some extreme examples, based on depends on tissue sensitivity, temperature and exposure time may result in tissue damage. A metric has been defined for implantable systems in which the time-temperature dose is quantified in terms of cumulative equivalent minutes at 43° C. (CEM43). ISO Standard 14708-3 (Implants for surgery, Active implantable medical devices, Part 3, Implantable neurostimulators: 2017) describes details for CEM43. This thermal dose model is based on studies that show an increase in cell death may occur when the tissue temperature exceeds 43° C. and that there is an exponential relationship with increasing temperature and linear relationship with time. In some examples, the system of this disclosure may control recharging session parameters, e.g., transmitted energy level, transmission time and similar parameters, based on estimates of CEM43. In other examples, such as in an electric vehicle, a different time-temperature dose relationship may be applicable that promotes the longevity of the battery of the vehicle and is considered safe for normal operation of the system.

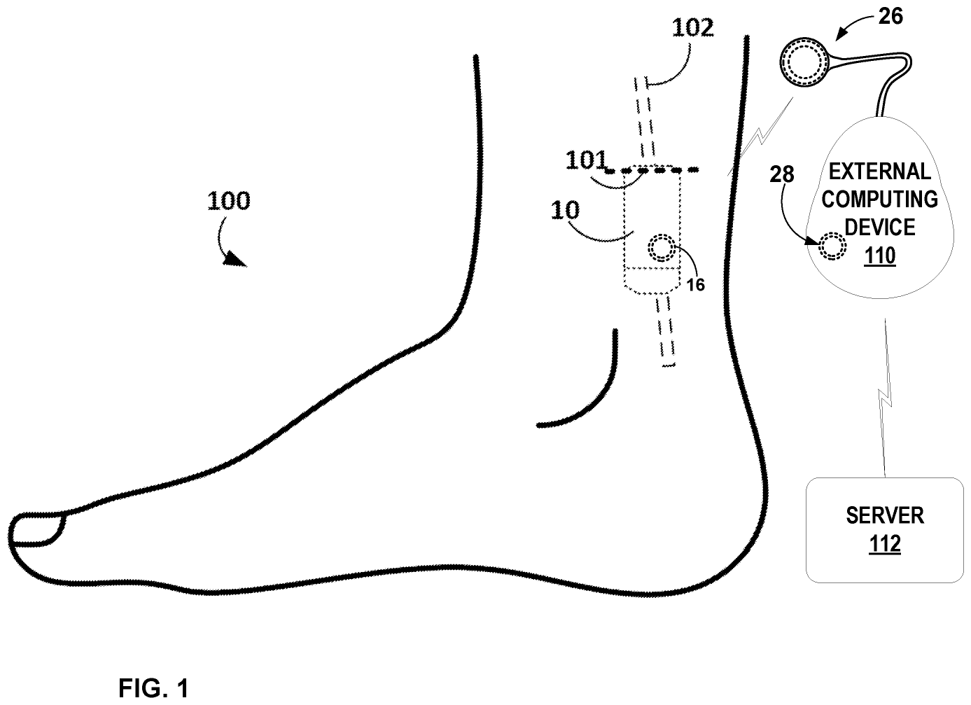

FIG. 1 is a conceptual diagram illustrating an example system including a medical device and external computing devices according to one or more techniques of this disclosure. The example of system 100 in FIG. 1 includes an implantable medical IMD 10, external computing device 110, and one or more servers 112.

External computing device 110 includes one or more antenna, such as antenna 26 and antenna 28. External computing device 110 may be used to program or adjust settings of IMD 10 and may also recharge an electrical energy storage device of IMD 10, such as a battery. External computing device 110 may also communicate with servers 112. In other examples, an external computing device separate from external computing device 110 may communicate with IMD 10 to adjust therapy and/or sensing parameters, download recorded data, and so on.

The example of FIG. 1 is a side view of a patient's leg showing IMD 10 near the ankle and adjacent to the tibial nerve 102. IMD 10 is a leadless neurostimulation device in the example of FIG. 1. IMD 10 can be implanted through the patient's skin and cutaneous fat layer via a small incision 101 (e.g., about one to three centimeters (cm)) above the tibial nerve on a medial aspect of the patient's ankle. While incision 101 is shown approximately horizontal to the length of the tibial nerve, other incisions or implantation techniques could be used according to physician preference. The example of FIG. 1 describes a neurostimulation implantable medical device for tibial nerve stimulation. In other examples, the techniques of this disclosure may apply to other medical devices, such as wearable or implantable neurostimulation system for use in spinal cord stimulation therapy (e.g., pain therapy), deep brain stimulation, pelvic floor stimulation (e.g., sacral nerve stimulation) as well as to other types of implantable or external medical devices without limitation.

In the example of FIG. 1, IMD 10 may be positioned adjacent to the region defined by flexor digitorum longus and soleus in which tibial nerve 102 is contained and implanted adjacent and proximal to a fascia layer. One or more electrodes of IMD 10 may face toward tibial nerve 102. Though not shown in FIG. 1, IMD 10 may also connect to one or more leads comprising one or more electrodes (not shown in FIG. 1).

IMD 10 may be constructed of any polymer, metal, or composite material sufficient to house the components of IMD 10. In this example, IMD 10 may be constructed with a biocompatible housing, such as titanium or stainless steel, or a polymeric material such as silicone or polyurethane, and surgically implanted at a site in patient near the tibial nerve, in some examples, while in other examples, implanted near the pelvis, abdomen, or buttocks. The housing of IMD 10 may be configured to provide a hermetic seal for components, such as a rechargeable power source. In addition, the housing of IMD 10 may be selected of a material that facilitates receiving energy to charge the rechargeable power source.

While providing therapy, an electrical stimulation signal may be transmitted between one or more electrodes through the fascia layer. The electrical signal may be used to stimulate tibial nerve 102 which may be useful in the treatment of overactive bladder (OAB) symptoms of urinary urgency, urinary frequency and/or urge incontinence, fecal incontinence, pain, or other symptoms. System 100 may help relieve some symptoms of some disorders.

One type of therapy for treating bladder dysfunction includes delivery of electrical stimulation to a target tissue site within a patient to cause a therapeutic effect during delivery of the electrical stimulation. For example, delivery of electrical stimulation from IMD 10 to a target therapy site, e.g., a tissue site that delivers stimulation to modulate activity of a tibial nerve, spinal nerve (e.g., a sacral nerve), a pudendal nerve, dorsal genital nerve, an inferior rectal nerve, a perineal nerve, or branches of any of the aforementioned nerves, may provide a therapeutic effect for bladder dysfunction, such as a desired reduction in frequency of bladder contractions. In some cases, electrical stimulation of the tibial nerve may modulate afferent nerve activities to restore urinary function.

In some examples, the techniques described in this disclosure are directed to delivery of neurostimulation therapy in a non-continuous manner which may include on-cycles and off-cycles. For example, an IMD may deliver neurostimulation therapy for a specified period of time followed by a specified period of time when the IMD does not deliver neurostimulation (e.g., withholds delivery of neurostimulation). A period during which stimulation is delivered (an on-cycle) may include on and off periods (e.g., a duty cycle or bursts of pulses) with short inter-pulse durations of time when pulses are not delivered.

The power source of IMD 10 may include one or more capacitors, batteries, or other components (e.g., chemical, or electrical energy storage devices). Example batteries may include lithium-based batteries, nickel metal-hydride batteries, or other materials. In some examples, the power source may be a primary cell battery that is replaced when depleted. In other examples, the power source may be rechargeable. The rechargeable power source may be replenished, refilled, or otherwise capable of increasing the amount of energy stored after energy has been depleted. The energy received from secondary coil 16 may be conditioned and/or transformed by a charging circuit. The charging circuit may then convey electrical energy to charge the rechargeable power source when the power source is fully depleted or only partially depleted.

External computing device 110 may be used to recharge the rechargeable power source within IMD 10 implanted in the patient. External computing device 110 may be a hand-held device, a portable device, or a stationary charging system. External computing device 110 may also be referred to as charging device 110 in this disclosure. External computing device 110 may include components necessary to charge IMD 10 through tissue of the patient. External computing device 110 may include a power transmitting antenna such as an internal energy transfer coil 28 and external energy transfer coil 26, also referred to as primary coil 26 or primary coil 28. In other examples, external computing device may only include internal primary coil 28 and omit the use of external primary coil 26, or only include external primary coil 26 and omit the use of internal primary coil 28.

External computing device 110 may include a housing to enclose operational components such as a processor, memory, user interface, telemetry module, power source, and charging circuit configured to transmit energy to secondary coil 16 via energy transfer coil 26 and/or 28. Although a user may control the recharging process with a user interface of external computing device 110, external computing device 110 may alternatively be controlled by another device, e.g., an external programmer, a computing device of servers 112 such as a tablet computer, laptop or other similar computing device. An external computing device of servers 112 may include a computing device with a touch-screen user interface. In other examples, external computing device 110 may be integrated with an external programmer, such as a patient programmer carried by the patient.

External computing device 110 may also be referred to as a power transmitting unit, or a power transmitting device. IMD 10 may be referred to as a power receiving unit or a power receiving device. External computing device 110 may include one or more processors, a driver circuit configured to cause the power transmitting antenna, e.g., primary coil 28, to transmit wireless electrical energy to IMD 10, as well sensors and other components (not shown in FIG. 1). For example, external computing device 110 may include first processing circuitry operatively coupled to a first memory. The first processing circuitry configured to execute programming instructions stored at the first memory to determine and output a target power magnitude. The first processing circuitry may determine the target power magnitude based on several factors. Some example factors may include the depth of discharge of the battery of IMD 10, a measured or calculated temperature of a primary coil and or of IMD 10, the coupling efficiency between primary coil 28 and secondary coil 16, the duration of the charging session and other factors.

Second processing circuitry, e.g., a second independent processor, may be configured to receive the target power magnitude from the primary processing circuitry and independently calculate the target power magnitude. The second processing circuitry may calculate the target power magnitude based on the same factors described above. In some examples, the second processing circuitry may compare the received target power magnitude to the independently calculated target power magnitude. If the values match, e.g., if the received and independently calculated values are approximately equal within a threshold range of each other, then external computing device 110 may begin delivering wireless power to IMD 10.

In some examples, the second processing circuitry may perform other verification checks of the first processing circuitry operation. For example, after determining the received target power magnitude matches the independently calculated target power magnitude, the second processing circuitry may compare the target power magnitude to a threshold output power limit. In an example in which the target power magnitude is outside the threshold output power limit, the second processing circuitry may control the driver circuit to terminate power transmission. The second processing circuitry may also output a signal that generates an alert, e.g., to the patient or to a caregiver via external computing device 110 and/or server 112.

In some examples, the first processing circuitry and the second processing circuitry may be implemented as microprocessors, which may include internal computer readable storage media. In some examples, the second processing circuitry may be a separate independent processor configured to perform verification and safety checks for the operation of external computing device 110, as well as other components of system 100. In other examples, the second processing circuitry may perform one or more other functions, in addition verifying the operation of the first processing circuitry. For example, the second processing circuitry may control the driver circuit for the power transmitting antenna, control communication circuitry, user interface functions and so on. In the example in which the second processing circuitry also controls the driver circuitry for the primary coil, the second processing circuitry may directly control the driver circuitry to deliver power or cease transmission, based on the results of the verification tests. In other examples, e.g., for an independent verification processor, the second processing circuitry may instead output a signal, e.g., a reset signal, a flag, and so on, that may cause the driver circuitry to stop delivering power.

In some examples, the secondary processing circuitry may also execute programming instructions to calculate an integral of power delivered over a predetermined time duration. The secondary processing circuitry may receive the measured power delivered from sensing circuitry, e.g., attached to or part of the power transmitting circuitry of external device 110. The secondary processing circuitry may compare the integral of the measured power delivered to an integral dose curve stored at a memory location accessible by the secondary processing circuitry. In response to determining that the calculated integral of power delivered exceeds the integral dose curve for the predetermined time duration, the second processing circuitry may control the driver circuit to terminate power transmission, as described above. In some examples,

In some examples, the second processing circuitry may also execute programming instructions implement a watchdog function. In other words, the secondary processing circuitry may check itself, using separate, independent programming instructions, e.g., stored at a memory location. The watchdog function instructions may cause the second processing circuitry to verify that, within a predetermined watchdog duration, the secondary processing circuitry independently calculated the target power magnitude and compared the received target power magnitude to the independently calculated target power magnitude. The second processing circuitry may operate one or more timers, including the watchdog duration timer. If the second processing circuitry does not run any one or more of the verification functions within the duration measured by the watchdog duration timer, the second processing circuitry may flag a watchdog failure.

External computing device 110 may take a variety of actions in response to such a watchdog failure. In one example, the secondary processing circuitry may control the driver circuitry to stop or reduce wireless power transfer. In other examples, the watchdog failure flag may cause a reset of external computing device 110. For example, the system within external computing device 110, e.g., the first processing circuitry, second processing circuitry, driver circuitry and other components of external computing device 110, may execute a reset, which may include restarting the system. In some examples, the secondary processing circuitry may output a signal that resets one or more components of external computing device 110.

In some examples, processing circuitry of servers 112 may include the second processing circuitry that performs the error checking described above. In some examples, processing circuitry of a smart phone, tablet computer or other similar device may include the second processing circuitry that performs the error checking functions, including the watchdog function.

External computing device 110 and IMD 10 may utilize any wireless power transfer techniques that are capable of recharging the power source of IMD 10 when IMD 10 is implanted within the patient. In some examples, system 100 may utilize inductive coupling between primary coils (e.g., energy transfer coil 28) and secondary coils (e.g., secondary coil 16) of external computing device 110 and IMD 10. In inductive coupling, energy transfer coil 28 is placed near implanted IMD 10 such that energy transfer coil 28 is aligned with secondary coil 16 of IMD 10. External computing device 110 may then generate an electrical current in energy transfer coil 28 based on a selected power level for charging the rechargeable power source of IMD 10. When the primary and secondary coils are aligned, the electrical current in the primary coil may magnetically induce an electrical current in secondary coil 16 within IMD 10. Since the secondary coil is associated with and electrically coupled to the rechargeable power source, the induced electrical current may be used to increase the voltage, or charge level, of the rechargeable power source. Although inductive coupling is generally described herein, any type of wireless energy transfer may be used to transfer energy between external computing device 110 and IMD 10.

Energy transfer coils 26 and 28 may include a wound wire (e.g., a coil) (not shown in FIG. 1). The coil may be constructed of a wire wound in an in-plane spiral (e.g., a disk-shaped coil). In some examples, this single or even multi-layers spiral of wire may be considered a flexible coil capable of deforming to conform with a non-planar skin surface. The coil may include wires that electrically couple the flexible coil to a power source and a charging module configured to generate an electrical current within the coil. Energy transfer coil 28 may be external of the housing of external computing device 110 such that energy transfer coil 28 can be placed on the skin of the patient proximal to IMD 10. In some examples, energy transfer coil 28 may be disposed on the outside of the housing or even within housing.

Either primary coil 26 and/or 28 of system 100 may include a heat sink device (not shown in FIG. 1). In the example of system 100, external computing device 110 is the power transmitting unit and IMD 10 is the power receiving unit. IMD 10 may be in a flipped or non-flipped position.

As noted above, in this disclosure external computing device 110 may also be referred to as recharger 110. External computing device 110 may include a user interface to receive control inputs from a user, such as the patient, medical professional, or other caregiver. The user interface of external computing device 110 may also provide information to a user. For example, external computing device 110 may include a button (not shown in FIG. 1) as well as a set of indicator lights configured to output information regarding communication status and wireless power transfer status.

External computing device 110 may receive wireless communication from IMD 10 that include the amount of power delivered to the electrical energy storage device of IMD 10, which may be referred to as closed loop charging. In other words, system 100 may measure efficiency, such as IMD efficiency, to determine whether the relative position of primary coil 26 and secondary coil 16 may be in a less desirable relative position. Processing circuitry of system 100, e.g., processing circuitry of external computing device 110, processing circuitry of servers 112, and/or processing circuitry of IMD 10, may calculate any of the values described herein.

Maximum Tank Power Profile

| Maximum Tank Power Profile (mW) | Aggregate Times (minutes) |
|---|---|
| 2850 | 0 to <15 |
| 2050 | 15 to <150 |
| 1550 | 150 to <320 |
| 0 | >=320 |

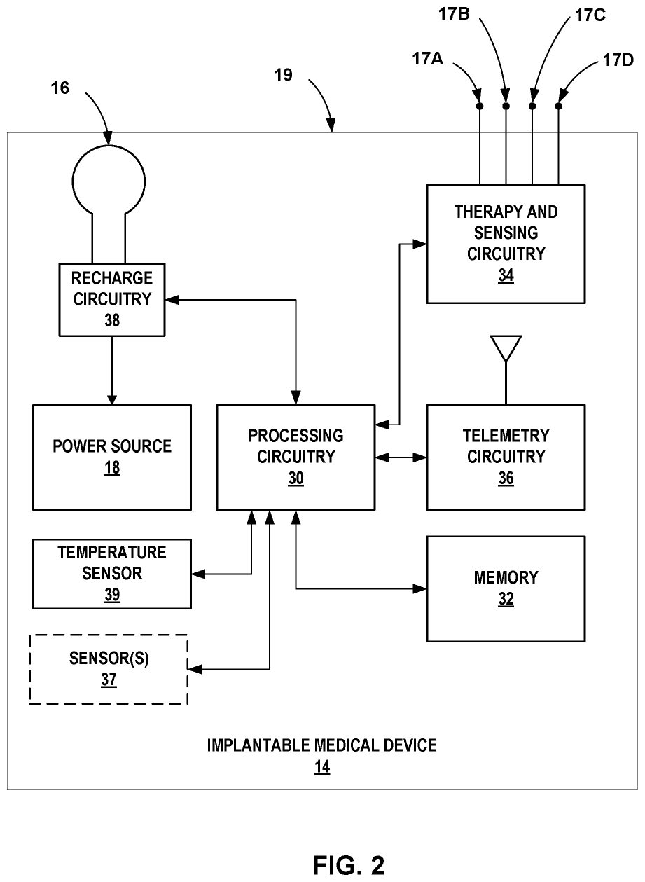

FIG. 2 is a block diagram illustrating example components of the medical device of FIG. 1. Implantable medical device 14 is an example of IMD 10 described above in relation to FIG. 1. In the example illustrated in FIG. 2, IMD 14 includes temperature sensor 39, coil 16, processing circuitry 30, therapy and sensing circuitry 34, recharge circuitry 38, memory 32, telemetry circuitry 36, power source 18, and one or more sensors 37, such as an accelerometer. In other examples, IMD 14 may include a greater or a fewer number of components, e.g., in some examples, IMD 14 may not include temperature sensor 39 or sensors 37. In some examples, power source 18 is a primary cell and not rechargeable, and the primary cell example, IMD 14 may not include recharge module 38. In general, IMD 14 may comprise any suitable arrangement of hardware, alone or in combination with software and/or firmware, to perform the various techniques described herein attributed to IMD 14 and processing circuitry 30, and any equivalents thereof.

Processing circuitry 30 of IMD 14 may include one or more processors, such as one or more microprocessors, digital signal processors (DSPs), application specific integrated circuits (ASICs), field programmable gate arrays (FPGAs), or any other equivalent integrated or discrete logic circuitry, as well as any combinations of such components. IMD 14 may include computer readable storage media, such as memory 32, which may be implemented as random access memory (RAM), read only memory (ROM), programmable read only memory (PROM), erasable programmable read only memory (EPROM), electronically erasable programmable read only memory (EEPROM), flash memory, comprising executable instructions for causing the processing circuitry 30 to perform the actions attributed to this circuitry. Moreover, although processing circuitry 30, therapy and sensing circuitry 34, recharge circuitry 38, telemetry circuitry 36, and temperature sensor 39 are described as separate modules, in some examples, some combination of processing circuitry 30, the circuitry include in therapy and sensing module 34, recharge circuitry 38, telemetry circuitry 36 and temperature sensor 39 are functionally integrated. In some examples, processing circuitry 30, therapy and sensing circuitry 34, recharge circuitry 38, telemetry circuitry 36, and temperature sensor 39 correspond to individual hardware units, such as ASICs, DSPs, FPGAs, or other hardware units. In this disclosure, therapy, and sensing circuitry 34 may be referred to as therapy circuitry 34 or therapy module 34, for simplicity.

Memory 32 may store therapy programs or other instructions that specify therapy parameter values for the therapy provided by therapy circuitry 34 and IMD 14. In some examples, memory 32 may also store temperature data from temperature sensor 39, instructions for recharging rechargeable power source 18, thresholds, instructions for communication between IMD 14 and an external computing device, or any other instructions required to perform tasks attributed to IMD 14. In various examples, memory 32 stores information related to determining the temperature of housing 19 and/or exterior surface(s) of housing 19 of IMD 14 based on temperatures sensed by one or more temperature sensors, such as temperature sensor 39, located within IMD 14.

In some examples, memory 32 may store programming settings such as electrical stimulation therapy output magnitude, pulse width, and so on. Memory 32 may determine whether a sensed bioelectrical signal is valid, such as and ECAP or other signal in response to an output electrical stimulation therapy event. Memory 32 may store programming instructions that when executed by processing circuitry 30 cause processing circuitry 30 to cause electrical stimulation circuitry therapy circuitry 34 to deliver electrical stimulation therapy to target tissue of a patient.

Therapy and sensing circuitry 34 may generate and deliver electrical stimulation under the control of processing circuitry 30. In some examples, processing circuitry 30 controls therapy circuitry 34 by accessing memory 32 to selectively access and load at least one of the stimulation programs to therapy circuitry 34. For example, in operation, processing circuitry 30 may access memory 32 to load one of the stimulation programs to therapy circuitry 34. In such examples, relevant stimulation parameters may include a voltage amplitude, a current amplitude, a pulse rate, a pulse width, a duty cycle, or the combination of electrodes 17A, 17B, 17C, and 17D (collectively “electrodes 17”) that therapy circuitry 34 may use to deliver the electrical stimulation signal as well as sense biological signals. In other examples, IMD 14 may have more or fewer electrodes than the four shown in the example of FIG. 2. In some examples electrodes 17 may be part of or attached to a housing of IMD 14, e.g., a leadless electrode. In other examples, one or more of electrodes 17 may be part of a lead implanted in or attached to a patient to sense biological signals and/or deliver electrical stimulation, as described above in relation to FIG. 1.

In some examples, one or more electrodes connected to therapy circuitry 34 may connect to one or more sensing electrodes, e.g., attached to the housing of IMD 14. In some examples the electrodes may be configured to detect the evoked motor response caused by the electrical stimulation therapy event, or other bioelectrical signals such as ECAPs, impedance and so on.

In the example of FIG. 2, IMD 14 also includes components to receive power to recharge rechargeable power source 18 when rechargeable power source 18 has been at least partially depleted. As shown in FIG. 2, IMD 14 includes coil 16 and recharge circuitry 38 coupled to rechargeable power source 18. Recharge circuitry 38 may be configured to charge rechargeable power source 18 with the selected power level determined by either processing circuitry 30 or an external charging device, such as external computing device 110 described above in relation to FIG. 1. Recharge circuitry 38 may include any of a variety of charging and/or control circuitry configured to process or convert current induced in coil 16 into charging current to charge power source 18.

Secondary coil 16 is an example of secondary coil 16 described above in relation to FIG. 1 and may include a coil of wire or other device capable of inductive coupling with a primary coil disposed external to patient 12. Although secondary coil 16 is illustrated as a simple loop of in FIG. 2, secondary coil 16 may include multiple turns of conductive wire. Secondary coil 16 may include a winding of wire configured such that an electrical current can be induced within secondary coil 16 from a magnetic field. The induced electrical current may then be used to recharge rechargeable power source 18.

Recharge circuitry 38 may include one or more circuits that process, filter, convert and/or transform the electrical signal induced in the secondary coil to an electrical signal capable of recharging rechargeable power source 18. For example, in alternating current induction, recharge circuitry 38 may include a half-wave rectifier circuit and/or a full-wave rectifier circuit configured to convert alternating current from the induction to a direct current for rechargeable power source 18. The full-wave rectifier circuit may be more efficient at converting the induced energy for rechargeable power source 18. However, a half-wave rectifier circuit may be used to store energy in rechargeable power source 18 at a slower rate. In some examples, recharge circuitry 38 may include both a full-wave rectifier circuit and a half-wave rectifier circuit such that recharge circuitry 38 may switch between each circuit to control the charging rate of rechargeable power source 18 and temperature of IMD 14.

Power source 18 may include one or more capacitors, batteries, and/or other energy storage devices. Power source 18 may deliver operating power to the components of IMD 14. In some examples, rechargeable power source 18 may include a power generation circuit to produce the operating power. Power source 18 may be configured to operate through many discharge and recharge cycles. Power source 18 may also be configured to provide operational power to IMD 14 during the recharge process. In some examples, rechargeable power source 18 may be constructed with materials to reduce the amount of heat generated during charging. In other examples, IMD 14 may be constructed of materials and/or using structures that may help dissipate generated heat at rechargeable power source 18, recharge circuitry 38, and/or secondary coil 16 over a larger surface area of the housing of IMD 14.

Although power source 18, recharge circuitry 38, and secondary coil 16 are shown as contained within the housing of IMD 14, in alternative implementations, at least one of these components may be disposed outside of the housing. For example, in some implementations, secondary coil 16 may be disposed outside of the housing of IMD 14 to facilitate better coupling between secondary coil 16 and the primary coil of an external charging device. In other examples, power source 18 may be a primary power cell and IMD 14 may not include recharge circuitry 38 and recharge coil 16, as noted above.

Processing circuitry 30 may also control the exchange of information with an external computing device using telemetry circuitry 36. Telemetry circuitry 36 may be configured for wireless communication using radio frequency protocols, such as BLUETOOTH, or similar RF protocols, as well as using inductive communication protocols. Telemetry circuitry 36 may include one or more antennas 37 configured to communicate with external charging device, for example. Processing circuitry 30 may transmit operational information and receive therapy programs or therapy parameter adjustments via telemetry circuitry 36. Also, in some examples, IMD 14 may communicate with other implanted devices, such as stimulators, control devices, or sensors, via telemetry circuitry 36. In addition, telemetry circuitry 36 may be configured to control the exchange of information related to sensed and/or determined temperature data, for example temperatures sensed by and/or determined from temperatures sensed using temperature sensor 39. In some examples, telemetry circuitry 36 may communicate using inductive communication, and in other examples, telemetry circuitry 36 may communicate using RF frequencies separate from the frequencies used for inductive charging.

In some examples, processing circuitry 30 may transmit additional information to external charging device related to the operation of rechargeable power source 18. For example, processing circuitry 30 may use telemetry circuitry 36 to transmit indications that rechargeable power source 18 is completely charged, rechargeable power source 18 is fully discharged, the amount of charging current output by recharge circuitry 38 e.g., to power source 18, or any other charge status of rechargeable power source 18. In some examples, processing circuitry 30 may use telemetry circuitry 36 to transmit instructions to external charging device, including instructions regarding further control of the charging session, for example instructions to lower the power level or to terminate the charging session, based on the determined temperature of the housing/external surface 19 of the IMD.

Processing circuitry 30 may also transmit information to external charging device that indicates any problems or errors with rechargeable power source 18 that may prevent rechargeable power source 18 from providing operational power to the components of IMD 14. In various examples, processing circuitry 30 may receive, through telemetry circuitry 36, instructions for algorithms, including formulas and/or values for constants to be used in the formulas that may be used to determine the temperature of the housing 19 and/or exterior surface(s) of housing 19 of IMD 14 based on temperatures sensed by temperature sensor 39 located within IMD 14 during and after a recharging session performed on rechargeable power source 18.

In some examples, processing circuitry 30 may act as the second independent processor, described above in relation to FIG. 1, and perform some of the error checking functions to check the operation of the first processing circuitry. For example, processing circuitry 30 may receive the target power magnitude, e.g., via telemetry circuitry 36, and independently calculate the target power magnitude. Processing circuitry 30 may also perform the comparisons of the target power magnitude to the calculated power magnitude, as well as to the threshold output power limit and the integrated power limit, as described above.

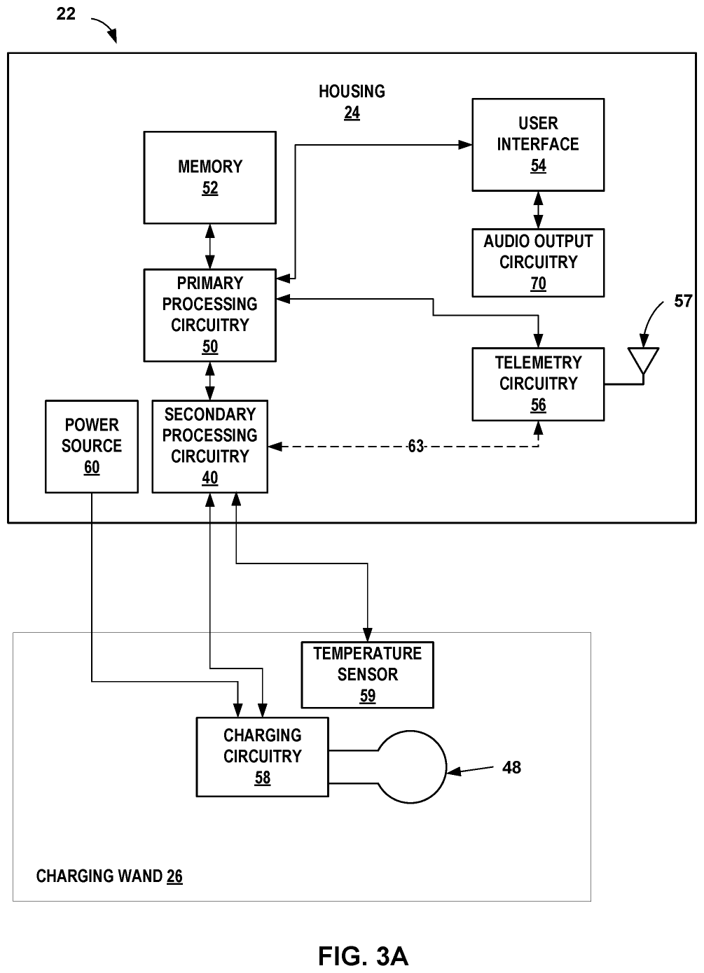

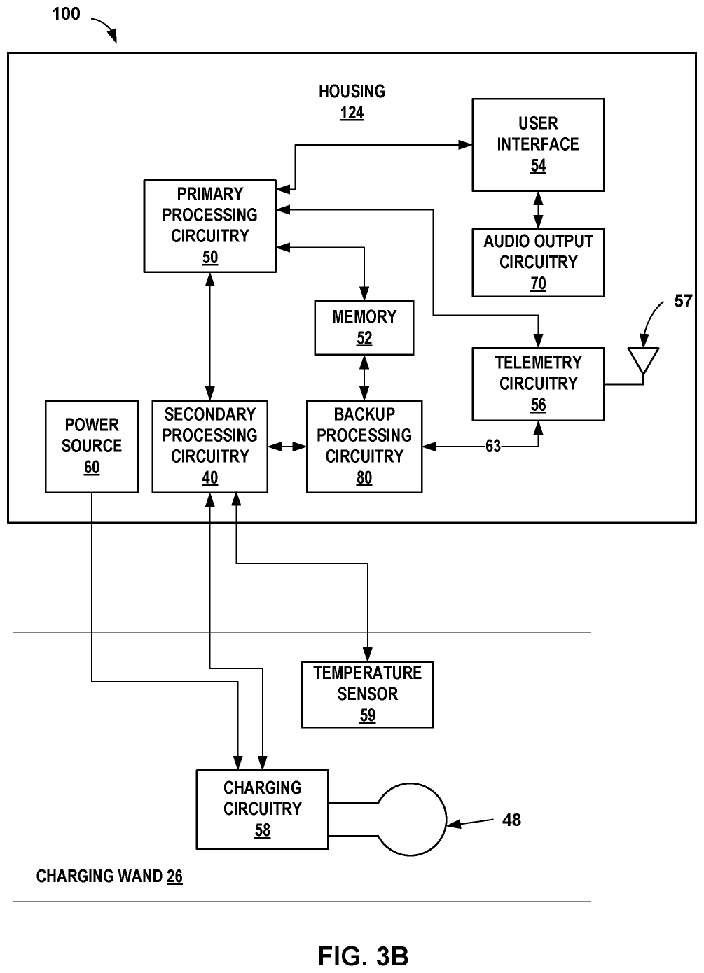

FIG. 3A is a block diagram of an example external computing device of FIG. 1. External charging device 22 in of FIG. 2 is an example of external computing device 110 described above in relation to FIG. 1. In some examples, external charging device 22 may be described as a hand-held device, in other examples, external charging device 22 may be a larger or a non-portable device. In addition, in other examples external charging device 22 may be included as part of an external programmer or include functionality of an external programmer. As shown in the example of FIG. 3A, external charging device 22 includes two separate components. Housing 24 encloses components such as a processing circuitry 50, memory 52, user interface 54, telemetry circuitry 56, audio output circuitry 70 and power source 60. Charging head 26, also called charging wand 26, may include charging circuitry 58, temperature sensor 59, and coil 48, which is an example of coil 28 described above in relation to FIG. 1.

In some examples, a separate charging wand 26 may facilitate positioning of coil 48 over coil 16 of IMD 14. In other examples, external charging device 22 may not include charging wand 26, and charging circuitry 58 and coil 48 may be integrated within housing 24, as described above in relation to FIG. 1. Memory 52 may store instructions that, when executed by processing circuitry 50, causes processing circuitry 50 and external charging device 22 to provide the functionality ascribed to external charging device 22 throughout this disclosure, and/or any equivalents thereof. Coil 28 may also be referred to as an antenna.

In some examples, external charging device 22 may include secondary processing circuitry 40. As described above in relation to FIG. 1, secondary processing circuitry 40 may execute programming instructions that include error checking of the operation of primary processing circuitry 50. In some examples, secondary processing circuitry 40 may also control charging circuitry 58, telemetry circuitry 56, user interface 54 or other functions of external charging device 22. In examples, primary processing circuitry 50 may control charging circuitry 58.

Secondary processing circuitry 40 may perform any one or more of several different error checking functions to independently verify the operation of primary processing circuitry 50. As described above in relation to FIG. 1, secondary processing circuitry 40 may receive a target power magnitude for charging circuitry 58 to drive a power transmitting antenna, e.g., coil 48 to output the wireless electrical energy. In some examples, secondary processing circuitry 40 may also independently calculate the target power magnitude and compare the received target power magnitude to the independently calculated target power magnitude.

In some examples, secondary processing circuitry 40 may receive information about the various factors used to calculate the second target power magnitude either directly or indirectly via primary processing circuitry 50. For example, secondary processing circuitry 40 may receive data from the power receiving unit, e.g., IMD 10 and IMD 14 described above in relation to FIGS. 1 and 2. Some examples of data from the power receiving unit may include an amount of charging current delivered to the battery of the IMD, temperature data from a temperature sensor in the IMD, a state of charge of the battery, e.g., fully charged, partially charged, and so on. Secondary processing circuitry 40 may also receive information such as the temperature of primary coil 48 from temperature sensor 59, a magnitude of current in primary coil 48, data to be used in estimating an amount of heating of the power receiving unit, an amount of time for a recharge session and other factors that may be used by primary processing circuitry 50 and secondary processing circuitry 40 to calculate the target power magnitude.

Along with comparing the received target power magnitude from primary processing circuitry 50 to the independently calculated target power magnitude, secondary processing circuitry 40 may determine whether received target power magnitude corresponds to, or matches the independently calculated target power magnitude. The received target power magnitude may correspond to, or match the independently calculated target power magnitude when the values are within a predetermined tolerance of each other, e.g., the difference between received target power magnitude and the independently calculated target power magnitude is less than a predetermined threshold value. In other words, that the received target power magnitude approximately equals the independently calculated target power magnitude. If the values do not match, secondary processing circuitry 40 may execute programming instructions to fail safe, e.g., to stop or reduce the output power, output an error flag, reset the system of external charging device 22, and so on. Secondary processing circuitry 40 may output signal that generates an alert, e.g., via user interface 54, telemetry circuitry 56, or audio output circuitry 70.

In some examples, the signal from secondary processing circuitry 40 that detects some fault, e.g., the target power exceeds a limit, the integral exceeds a limit or some other fault, may cause the driver circuit to stop output power for a predetermined stop duration. In other examples, primary processing circuitry 50 may cause the output power to stop during normal operation, such as the battery for the IMD is full, the user pushes button to stop charging, the user removes the external charging device from the patient's body and processing circuitry 50 times out the connection thereby stopping as well as for a fault detected by primary processing circuitry 50.

Secondary processing circuitry 40 may perform other error checks in addition to or instead of determining the received target power magnitude matches the independently calculated target power magnitude. In some examples, secondary processing circuitry 40 may compare the target power magnitude to a threshold output power limit. The threshold output power limit may be a single value, a range of values, a value that changes based on the length of time of the recharge session, may be a value in a lookup table stored, for example at a memory location for secondary processing circuitry 40 (not shown in FIG. 3A) or at memory 52. The threshold output power limit may also be a calculated value based on taking into account one or more factors described above, e.g., time, a calculated or measured amount of heat delivered to the power receiving unit and so on. Secondary processing circuitry may determine the threshold output power limit, e.g., the maximum allowed output power for a particular time period of the charging session, by any of the above techniques, e.g., lookup table, calculation, and similar techniques. This error check may prevent the correct power from being provided for too long.

As described above in relation to FIG. 1, secondary processing circuitry 40 may also calculate an integral of power delivered over a predetermined time duration. Secondary processing circuitry 40 may receive the information for the measured powered delivered (e.g., Ptank-measured) either directly from charging circuitry 58 or indirectly via primary processing circuitry 50. The calculation of measured power may include the temperature received from temperature sensor 59. This error check may detect both excessive power for the right duration and the correct target power for too long of a duration. Secondary processing circuitry 40 may compare the integral of power delivered to an integral dose curve stored at a memory location accessible by the second processing circuitry. In response to determining that the calculated integral of power delivered exceeds the integral dose curve for the predetermined time duration, secondary processing circuitry 40 may control the driver circuit to terminate power transmission, or take some other action to fail safe. Secondary processing circuitry 40 may also check itself by executing separate programming instructions for a watchdog function, as described above in relation to FIG. 1.

In some examples, the calculation for the integral of power delivered may be based on Ptank-measured multiplied by the amount of time since the last integral time step. It may be desirable to get frequent updates for the Ptank measurement such as every second or several times per second. In the example of a four Hertz measurement cycle, the amount of time since the last integral time step is 250 msec. For startup, or in examples in which external charging device 22 has restarted because of a pause in power delivery, then the processing circuitry may use a default time, e.g., 250 msec, rather than the time since the last power delivered measurement.

In other examples, secondary processing circuitry 40 may receive information from the IMD independent from primary processing circuitry 50. For example, rather than receiving data received by telemetry circuitry 56 via primary processing circuitry 50, secondary processing circuitry 40 may receive data sent by the IMD directly from telemetry circuitry 56, e.g., via connection 63. In the example in which the IMD communicates with external charging device 22 via inductive communication via coil 48, secondary processing circuitry 40 may receive data sent by the IMD through charging circuitry 58. In this manner, secondary processing circuitry 40 may independently check the estimate of heat in the IMD even with a malfunction in primary processing circuitry 50 that affects data transfer from telemetry circuitry 56.

In some examples, secondary processing circuitry 40 may independently estimate the heating and/or temperature of the IMD using a reduced order model, for example a first order or second order low pass filter from the heat inputs, e.g., received via telemetry circuitry 56, e.g., as described by U.S. Pat. No. 11,495,987 entitled WIRELESS RECHARGING DEVICES AND METHODS BASED ON THERMAL BOUNDARY CONDITIONS, assigned to Medtronic, Inc. In the example in which the IMD includes one or more temperature sensors 39, described above in relation to FIG. 1, secondary processing circuitry 40 may receive temperature data from one or more locations of the IMD. Secondary processing circuitry 40 may receive other heat control related information from the IMD, such as IMD battery voltage, battery current and similar information. Secondary processing circuitry 40 may independently estimate the heating for the IMD using the same process as primary processing circuitry 50, based on the received heat control information. For example, both primary processing circuitry 50 and secondary processing circuitry 40 may determine the heating of the IMD (Q_{IMD}) based on:

P_{IMD_batt}=I_{IMD_batt}V_{IMD_batt}−I_{IMD_batt}²R_{IMD_batt} [1]

Q_{IMD}=Pₜₐₙₖ−Qₚᵣᵢₘ−P_{IMD_batt} [2]

where R_{IMD_batt} is based on characteristics of the power receiving device.

In some examples, secondary processing circuitry 40 may estimate CEM43, as described above in relation to FIG. 1, and take some action to fail safe, as described above, e.g., secondary processing circuitry 40 may compare the estimated CEM43 to one or more thresholds and take an action based on the comparison. For example, secondary processing circuitry 40 may pause or terminate recharging, output a signal to reduce the recharge power, generate an alert, or other similar actions.

$$CEM 43 = {\sum }_{i = 1}^{n} {t}_{i} \times {R}^{( 43 - {T}_{i} )} \qquad [ 3 ]$$

where:
CEM43=the cumulative number of equivalent minutes at 43° C., as described above.
R is a constant related to the temperature dependence on cell damage:
   R=0.25 for T<43° C.;
   R=0.5 for T≥43° C.;
tᵢ=the duration of a time interval
Tᵢ=the average temperature over the respective time interval

External charging device 22 may also include one or more temperature sensors, illustrated as temperature sensor 59, similar to temperature sensor 39 of FIG. 2. As shown in FIG. 3A, temperature sensor 59 may be disposed within charging head 26. In other examples, one or more temperature sensors of temperature sensor 59 may be disposed within housing 24. For example, charging head 26 may include one or more temperature sensors positioned and configured to sense the temperature of coil 48 and/or a surface of the housing of charging head 26. In some examples, external charging device 22 may not include temperature sensor 59.

In general, external charging device 22 comprises any suitable arrangement of hardware, alone or in combination with software and/or firmware, to perform the techniques ascribed to external charging device 22, and processing circuitry 50, user interface 54, telemetry circuitry 56, and charging circuitry 58 of external charging device 22, and/or any equivalents thereof. In various examples, external charging device 22 may include one or more processors, such as one or more microprocessors, DSPs, ASICs, FPGAs, or any other equivalent integrated or discrete logic circuitry, as well as any combinations of such components. External charging device 22 also, in various examples, may include a memory 52, such as RAM, ROM, PROM, EPROM, EEPROM, flash memory, a hard disk, a CD-ROM, comprising executable instructions for causing the one or more processors to perform the actions attributed to them. Moreover, although processing circuitry 50, telemetry circuitry 56, charging circuitry 58, and temperature sensor 59 are described as separate modules, in some examples, processing circuitry 50, telemetry circuitry 56, charging circuitry 58, and/or temperature sensor 59 are functionally integrated. In some examples, processing circuitry 50, telemetry circuitry 56, charging circuitry 58, and/or temperature sensor 59 correspond to individual hardware units, such as ASICs, DSPs, FPGAs, or other hardware units.

Memory 52 may store instructions that, when executed by processing circuitry 50, cause processing circuitry 50 and external charging device 22 to provide the functionality ascribed to external charging device 22 throughout this disclosure, and/or any equivalents thereof. For example, memory 52 may include instructions that cause processing circuitry 50 to control the power level used to charge IMD 14 in response to the determined temperatures for the housing/external surface(s) of IMD 14, as communicated from IMD 14, or instructions for any other functionality. Memory 52 may include a record of selected power levels, sensed temperatures, determined temperatures, or any other data related to charging rechargeable power source 18, described above in relation to FIG. 2.

Processing circuitry 50 may, when requested, transmit any stored data in memory 52 to another computing device for review or further processing, such as to server 112 depicted in FIG. 1. Processing circuitry 50 may be configured to access memory, such as memory 32 of IMD 14 and/or memory 52 of external charging device 22, to retrieve information comprising instructions, formulas, and determined values for one or more constants.

User interface 54 may include buttons, a keypad, lights, such as indicator lights, a speaker for voice commands, a display, such as a liquid crystal (LCD), light-emitting diode (LED), or cathode ray tube (CRT). In some examples, the display may be a touch screen. As discussed in this disclosure, processing circuitry 50 may present and receive information relating to the charging of rechargeable power source 18 via user interface 54. For example, user interface 54 may indicate when charging is occurring, quality of the alignment between primary coil 28 or 48 and the secondary coil of the IMD, the selected power level, current charge level of rechargeable power source 18, duration of the current recharge session, anticipated remaining time of the charging session, sensed temperatures, or any other information. Processing circuitry 50 may receive some of the information displayed on user interface 54 from IMD 14 in some examples. In some examples, user interface 54 may provide an indication to the user regarding the quality of alignment between coil 16, depicted in FIG. 2 and coil 48, based on the charge current to the battery.

User interface 54 may also receive user input via user interface 54. The input may be, for example, in the form of pressing a button on a keypad or selecting an icon from a touch screen. The input may change programmed settings, start, or stop therapy, request starting or stopping a recharge session, a desired level of charging, or one or more statistics related to charging rechargeable power source 18 (e.g., the cumulative thermal dose). In this manner, user interface 54 may allow the user to view information related to the operation of IMD 14.

Charging circuitry 58 may include one or more circuits that generate an electrical signal, and an electrical current, within primary coil 48. Charging circuitry 58 may generate an alternating current of specified amplitude and frequency in some examples. In other examples, charging circuitry 58 may generate a direct current. In any case, charging circuitry 58 may be capable of generating electrical signals, and subsequent magnetic fields, to transmit various levels of power to IMD 14. In this manner, charging circuitry 58 may be configured to charge rechargeable power source 18 of IMD 14 with the selected power level.

Power source 60 may deliver operating power to the components of external charging device 22. Power source 60 may also deliver the operating power to drive primary coil 48 during the charging process. Power source 60 may include a battery and a power generation circuit to produce the operating power. In some examples, a battery of power source 60 may be rechargeable to allow extended portable operation. In other examples, power source 60 may draw power from a wired voltage source such as a consumer or commercial power outlet.

Telemetry circuitry 56 supports wireless communication between IMD 14 and external charging device 22 under the control of processing circuitry 50. Telemetry circuitry 56 may also be configured to communicate with another computing device via wireless communication techniques, or direct communication through a wired connection. In some examples, telemetry circuitry 56 may be substantially similar to telemetry circuitry 36 of IMD 14 described herein, providing wireless communication via an RF or proximal inductive medium. In some examples, telemetry circuitry 56 may include an antenna 57, which may take on a variety of forms, such as an internal or external antenna. Although telemetry circuitry 56 and 36 may each include dedicated antennas for communications between these devices, telemetry circuitry 56 and 36 may instead, or additionally, be configured to utilize inductive coupling from coils 16 and 48 to transfer data.

Examples of local wireless communication techniques that may be employed to facilitate communication between external charging device 22 and IMD 14 include radio frequency and/or inductive communication according to any of a variety of standard or proprietary telemetry protocols, or according to other telemetry protocols such as the IEEE 802.11x or Bluetooth specification sets. In this manner, other external devices may be capable of communicating with external charging device 22 without needing to establish a secure wireless connection.

As described above in relation to FIG. 1, primary processing circuitry 50 may use any one or more system metrics to determine power transfer to IMD 10. In some examples, IMD 10 may send a signal indicating an amount of current output by the recharge circuitry of IMD 10. In other examples, primary processing circuitry 50 may calculate other system metrics, such as alignment of coil 48 to coil 16 of IMD 10 using any of several techniques, including heat calculations, temperature measurements, detection of metal, and so on. Primary processing circuitry 50 may compare any of the calculated power transfer, power efficiency, alignment, IMD 10 current, etc. to a threshold stored at memory 52. Primary processing circuitry 50 may use the power transfer, and other metrics, to calculate the target output power.

FIG. 3B is a block diagram of an example external computing device of FIG. 1 that include a backup processor. External computing device 100 is an example of external computing device 22 described above in relation to FIG. 3A. Components in external computing device 100 with the same reference numbers as in external computing device 22 have the same or similar functions and characteristics. This description of FIG. 3B may include only the differences between FIGS. 3A and 3B. As shown in the example of FIG. 3A, external charging device 22 includes two separate components. Housing 124 encloses components such as a processing circuitry 50, memory 52, user interface 54, telemetry circuitry 56, audio output circuitry 70 and power source 60. Charging head 26, also called charging wand 26, may include charging circuitry 58, temperature sensor 59, and coil 48, which is an example of coil 28 described above in relation to FIG. 1.

In addition, external computing device 100 includes backup processing circuitry 80. Backup processing circuitry 80 may receive the same or similar information as does primary processing circuitry 50 to calculate the target output power for a period of time during a charging session. As with primary processing circuitry 50, backup processing circuitry 80 may receive an indication of the measured output power (e.g., Ptank-measured) from secondary processing circuitry 40. Backup processing circuitry 80 may also directly receive information from the wireless power receiving device, e.g., IMD 10 of FIG. 1, from telemetry circuitry 56, such as the battery current, state of charge of the IMD battery and similar information described above in relation to FIGS. 1-3A. In this manner, backup processing circuitry 80 may independently calculate the target output power and output the second calculation of the target output power to secondary processing circuitry 40.

In the example of FIG. 3B, primary processing circuitry 50 and backup processing circuitry 80 both operatively connect to memory 52. Memory 52 may include programming instructions, data, operating parameters used to execute the functions described herein. In other examples, each of primary processing circuitry 50 and backup processing circuitry 80 both operatively connect to separate memory (not shown in FIG. 3B). In other examples, each of primary processing circuitry 50 and backup processing circuitry 80 may include an internal memory (not shown in FIG. 3B), such as found in a system on chip (SOC), a microprocessor or similar implementations of processing circuitry.

Secondary processing circuitry 40 may receive the target power magnitude from both primary processing circuitry 50 and backup processing circuitry 80. Secondary processing circuitry 40 may compare the received first and second target power magnitudes. If both target power magnitudes match, e.g., within a threshold range of each other, secondary processing circuitry 40 may proceed to output the wireless power according to the received target power magnitude. In some examples, secondary processing circuitry may also perform the output power threshold comparison and integral power dose comparison described above in relation to FIGS. 1 and 3A. In other examples, backup processing circuitry 80, or some combination of system processing circuitry, may perform the output power threshold comparison and integral power dose comparison instead of, or along with the comparison performed by secondary processing circuitry 40. Similarly, either or any combination of backup processing circuitry 80 and secondary processing circuitry 40 may perform the other checks described above, such as the watchdog check.

If both target power magnitudes do not match, then the processing circuitry may take any one or more of several actions. One example action may be to flag an error and stop the wireless power transfer. Another example action may be to deliver wireless power at the lower of the two received target power magnitudes, along with the other actions described above in relation to FIG. 3A.

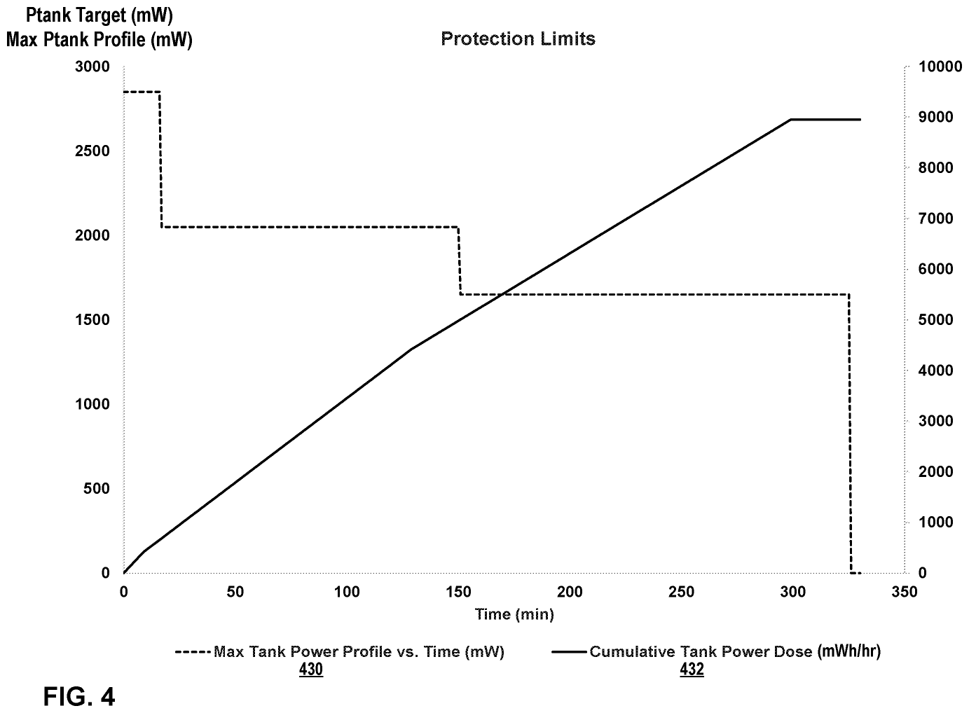

FIG. 4 is a graph illustrating example threshold output power profile limits and integral dose curve limits according to one or more techniques of this disclosure. As described above in relation to FIGS. 1-3, the secondary processing circuitry may independently calculate the target power magnitude and compare the target power magnitude to a threshold output power limit. The secondary processor may also compare the integral of power delivered to an integral dose curve. FIG. 4 illustrates an example threshold power magnitude profile, e.g., maximum tank power profile 430 and an example integral dose curve, e.g., cumulative tank power dose 432. As shown in the example of FIG. 4, the threshold values of both the maximum tank power profile 430 and the cumulative tank power dose 432 may change over time as described above in relation to the tables in the description of FIG. 1.

The example threshold power magnitude profile, maximum tank power profile 430 in FIG. 4 is a step-wise continuous function. The example of FIG. 4 may also be described by a step table, e.g., a first value of about 2700 mW from 0-25 minutes, a second value of 2000 mW from 25-150 minutes and so on. However, in other examples, threshold power magnitude profile may be a constant, or may change over time based on a linear or non-linear relationship, and so on. In the example of FIG. 4, the duration of the wireless power transfer session is limited to about 325 minutes. In other examples, the duration of the wireless power transfer session may be limited to a different value.

Comparing the target power magnitude to a threshold output power limit, using the maximum commanded target power, may prevent the correct power from being provided for too long. In other words, the target power magnitude may be correct, in that both the primary processing circuitry and secondary processing circuitry arrive at matching values for the target power magnitude. But the threshold output power limit may restrict power settings based on the length of the recharge session. For example, as shown in FIG. 4, the target power magnitude may be set as high as 2000 mW, but only for the first 150 minutes of the recharging session. A target power magnitude value of 2000 mW after 150 minutes may be outside the threshold output power limit, and the secondary processing circuitry may take some action such as control the driver circuit to reduce or terminate power transmission.

The secondary processing circuitry may also calculate an integral of power delivered, based on the measured tank power (Ptank-measured) over a predetermined time duration, for example, a time duration from the start of the recharge session. In other words, calculate the integral of power delivered based on a measured power delivered by the power transmitting unit. Cumulative tank power dose curve 432 is an integral dose curve that may be stored at a memory location accessible by the secondary processing circuitry. At a time selected by programming instructions stored at memory, the secondary processing circuitry may compare the calculated integral of power delivered by the power transmitting unit to an integral dose curve such as cumulative tank power dose curve 432. In other examples, the secondary processing circuitry may receive an indication from the power receiving device, e.g., IMD 14 via telemetry circuitry 36, described above in relation to FIG. 2, of the amount of power received. The secondary processing circuitry may calculate the integral of power received based on the data from the IMD.

Verifying the calculated integrated power dose to the integral dose curve may detect an output power magnitude that exceeds a threshold for an expected charging session duration. Verifying the integrated power dose may also detect a correct output power magnitude but delivered for a longer than expected duration. In response to determining that the calculated integral of power delivered exceeds the integral dose curve for the predetermined time duration, the secondary processing circuitry may control the driver circuit to terminate power transmission or take some other action, as described above.

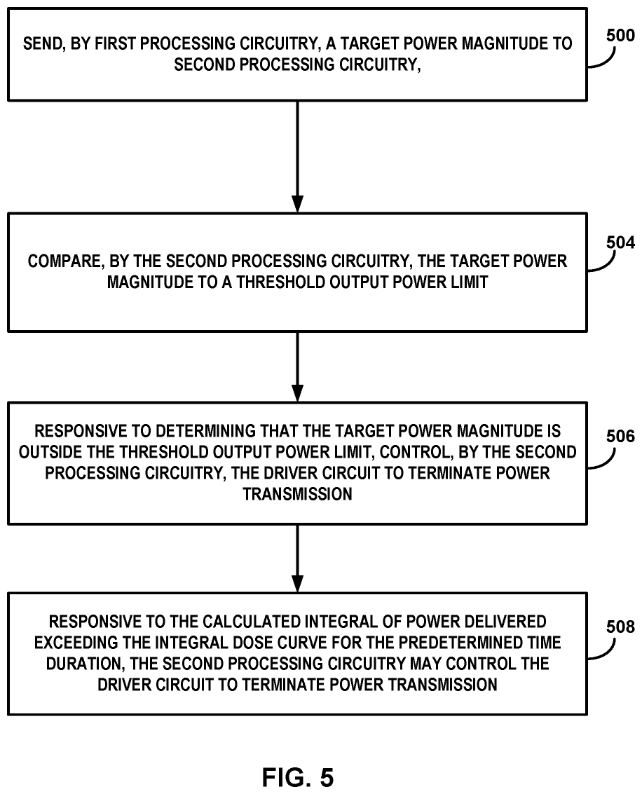

FIG. 5 is a flow diagram illustrating an example operation for delivering wireless power, in accordance with one or more techniques of this disclosure. The example technique described in FIG. 5 is described with respect to the components of external computing device 22, but other devices may similarly perform the described functions. As seen in the example of FIG. 5, first processing circuitry 50 initially may send, a target power magnitude to second processing circuitry (500). The second processing circuitry, e.g., secondary processing circuitry 40, may be configured to control a driver circuit, e.g., charging circuitry 58, that may drive power transmitting antenna 48 to transmit wireless electrical energy based on the target power magnitude, as described above in relation to FIGS. 1-4. The wireless electrical energy may provide wireless power to an implantable medical device, e.g., IMD 10 and IMD 14 depicted in FIGS. 1 and 2. In other examples, secondary processing circuitry 40 may be configured to perform other functions of external computing device 22 or to only perform error checking functions. In other examples, IMD 10 may include backup processing circuitry 80 that performs error checking functions in addition to, or instead of, secondary processing circuitry 40, as described above in relation to FIG. 3B.

In some examples, second processing circuitry 40 may compare the received target power magnitude to the independently calculated target power magnitude. Second processing circuitry 40 may independently calculate the target power magnitude in example implementations where second processing circuitry 40 also receives the same or similar information as primary processing circuitry 50, e.g., telemetry information from the IMD. Further, second processing circuitry 40 may compare the target power magnitude to a threshold output power limit, e.g. maximum tank power profile 430 depicted in FIG. 4 (504). In some examples, second processing circuitry 40 may calculate the threshold output power limit. In other examples, second processing circuitry 40 may retrieve the threshold output power limit from a lookup table.

Responsive to determining that the target power magnitude is outside the threshold output power limit, e.g., exceeds the maximum allowable power limit, the second processing circuitry 40 may control the driver circuit to terminate power transmission (506), reduce power transmission, flag an error, or take some other action. In other words, if the calculated target power magnitude is above the threshold at the specified time, e.g., after 200 minutes of charging, the target power magnitude is greater than 1500 mW, in the example of FIG. 4, the secondary processing circuitry 40 may take some action to reduce or stop power transmission.

In some examples, secondary processing circuitry 40 may also calculate an integral of power delivered over a predetermined time duration, as described above in relation to FIGS. 3 and 4. Responsive to the calculated integral of power delivered exceeding the integral dose curve, e.g., cumulative tank power dose 432, for the predetermined time duration, secondary processing circuitry 40 may control the driver circuit to terminate or otherwise reduce power transmission (508).

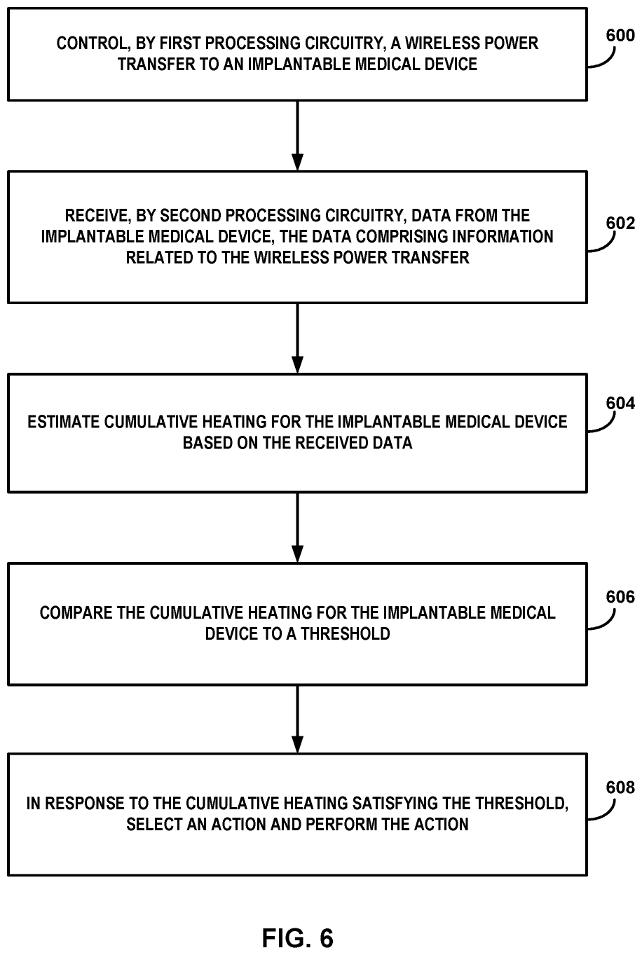

FIG. 6 is a flowchart illustrating an example operation of the secondary processing circuitry of this disclosure. As with FIG. 5, the example technique described in FIG. 6 is described with respect to the components of external computing device 22 of FIG. 3, but other devices may similarly perform one or more of the described functions.

First processing circuitry, e.g., primary processing circuitry 50, may execute programming instructions to control the wireless power transfer to an implantable medical device, by controlling charging circuitry 58 (600). Secondary processing circuitry 40, may receive data from the implantable medical device (602). In some examples secondary processing circuitry 40 may receive the data, e.g., data comprising information related to the wireless power transfer, via primary processing circuitry 50. In other examples, as described above in relation to FIG. 3, secondary processing circuitry 40 may receive temperature, current, battery voltage and other data from the IMD directly from telemetry circuitry 56 and/or charging circuitry 58.

Secondary processing circuitry 40 may execute programming instructions stored at a memory location to estimate the cumulative heating for the implantable medical device based on the received data (604). In some examples, secondary processing circuitry 40 may calculate the cumulative heating as CEM43, described above in relation to FIG. 3. In other examples, the cumulative heating may be based on a cumulative temperature curve. In some examples the computer readable storage medium from which the secondary processing circuitry retrieves the program instructions may be a separate computer readable storage medium, e.g., a separate memory unit, than the memory unit storing the programming instructions for the first processing circuitry. For example, primary processing circuitry 50 may retrieve programming instructions from memory 52, while secondary processing circuitry 40 may retrieve programming instructions, either from memory 52, or in other examples, from a memory internal to secondary processing circuitry 40, or a separate memory unit (not shown in FIG. 3A or 6.

Secondary processing circuitry 40 may compare the cumulative heating for the implantable medical device to one or more thresholds (606). In response to the cumulative heating satisfying one of the thresholds, secondary processing circuitry 40 may select an action. For example, in response to the cumulative heating exceeding a first threshold, secondary processing circuitry 40 may output an alert and/or reduce the output power. In response to the cumulative heating exceeding a second threshold with higher heating and/or higher temperature, secondary processing circuitry 40 may terminate power output, or temporarily pause power output for a specified duration.

The techniques of this disclosure are also disclosed by the following examples.

Example 1: A medical system comprising a power transmitting unit comprising a power transmitting antenna; a driver circuit configured to cause the power transmitting antenna to output wireless electrical energy to be received by an implantable medical device; first processing circuitry operatively coupled to a first memory, the first processing circuitry configured to execute programming instructions stored at the first memory to determine and output a first target power magnitude; and second processing circuitry configured to: control the driver circuit; receive the first target power magnitude; compare the first target power magnitude to a threshold output power limit; and responsive to determining that the first target power magnitude is outside the threshold output power limit, control the driver circuit to terminate power transmission.

Example 2: The medical system of example 1, wherein the threshold output power limit is a value on an output power profile, wherein the output power profile comprises a plurality of output power thresholds over a duration of a wireless power transfer session.

Example 3: The medical system of any of examples 1 and 2, wherein the second processing circuitry is further configured to: calculate an integral of power delivered over a predetermined duration; compare the integral of power delivered to an integral dose curve stored at a memory location accessible by the second processing circuitry; and responsive to determining that the calculated integral of power delivered exceeds the integral dose curve for the predetermined time duration, control the driver circuit to terminate power transmission.

Example 4: The medical system of example 3, wherein the power delivered over the predetermined duration is based on a measured power delivered by the power transmitting unit.

Example 5: The medical system of any of examples 3 and 4, wherein responsive to the driver circuit stopping the output power for a predetermined stop duration, the second processing circuitry is configured to reset the integration calculation of power delivered.

Example 6: The medical system of any of examples 1 through 5, wherein the secondary processing circuitry is further configured to: independently calculate a second target power magnitude; compare the first target power magnitude to the second target power magnitude independently calculated by the second processing circuitry; responsive to determining that the first target power magnitude fails to correspond to the second target power magnitude, control the driver circuit to fail safe: wherein to fail safe comprises to terminate power transmission, and wherein to correspond to the second power comprises that the second target power magnitude is within a predetermined tolerance of the first target magnitude.

Example 7: The medical system of any of examples 1 through 6, further comprising independently calculate a second target power magnitude; output the second target power magnitude to the second processing circuitry.

Example 8: The medical system of example 7, wherein the second processing circuitry is further configured to: receive the second target power magnitude; compare the first target power magnitude to the second target power magnitude independently calculated by the second processing circuitry; responsive to determining that the first target power magnitude fails to correspond to the second target power magnitude, control the driver circuit to fail safe: wherein to fail safe comprises to terminate power transmission, and wherein to correspond to the second power comprises that the second target power magnitude is within a predetermined tolerance of the first target magnitude.

Example 9: The medical system of any of examples 7 and 8, wherein the second processing circuitry is further configured to: receive the second target power magnitude; compare the first target power magnitude to the second target power magnitude independently calculated by the second processing circuitry; responsive to determining that the first target power magnitude fails to correspond to the second target power magnitude, control the driver circuit to output wireless electrical energy based on the lower of the first target power magnitude and the second target power magnitude.

Example 10: The medical system of any of examples 1 through 9, wherein the second processing circuitry is further configured to execute a watchdog function, wherein the watchdog function causes the second processing circuitry to verify that, within a predetermined watchdog duration, the second processing circuitry independently calculated the target power magnitude and compared the received target power magnitude to the independently calculated target power magnitude, responsive to the watchdog duration expiring before the second processing circuitry independently calculated the target power magnitude and compared the received target power magnitude to the threshold output power limit, the watchdog function causes the second processing circuitry to output a signal that resets the power transmitting unit.

Example 11: The medical system of any of examples 1 through 10, further comprising the implantable medical device configured to receive at least a portion of the wireless electrical energy.

Example 12: A power transmitting device comprising a power transmitting antenna; a driver circuit configured to cause the power transmitting antenna to transmit wireless electrical energy to an implantable medical device; first processing circuitry operatively coupled to a first memory, the first processing circuitry configured to execute programming instructions stored at the first memory to determine and output a first target power magnitude; and second processing circuitry configured to: control the driver circuit; receive the first target power magnitude; compare the first target power magnitude to a threshold output power limit; and responsive to determining that the first target power magnitude is outside the threshold output power limit, control the driver circuit to terminate power transmission.

Example 13: The power transmitting device of example 12, wherein the threshold output power limit is a value on an output power profile, wherein the output power profile comprises a plurality of output power thresholds over a duration of a wireless power transfer session.

Example 14: The power transmitting device of any of examples 12 and 13, wherein the second processing circuitry is further configured to: calculate an integral of power delivered over a predetermined duration; compare the integral of power delivered to an integral dose curve stored at a memory location accessible by the second processing circuitry; and responsive to determining that the calculated integral of power delivered exceeds the integral dose curve for the predetermined time duration, control the driver circuit to terminate power transmission.

Example 15: The power transmitting device of example 14, wherein the power delivered over the predetermined duration is based on a measured power delivered by the power transmitting unit.

Example 16: The power transmitting device of any of examples 14 and 15, wherein responsive to driver circuit stopping the output power for a predetermined stop duration, the second processing circuitry is configured to reset the integration calculation of power delivered.

Example 17: The power transmitting device of any of examples 12 through 16, wherein the second processing circuitry is further configured to execute a watchdog function, and wherein the watchdog function causes the second processing circuitry to verify that, within a predetermined watchdog duration, the second processing circuitry independently calculates the target power magnitude and compares the received target power magnitude to the threshold output power limit. Responsive to the watchdog duration expiring before the second processing circuitry independently calculates the target power magnitude and compares the received target power magnitude to the independently calculated target power magnitude, the watchdog function causes the second processing circuitry to output a signal that resets the power transmitting unit.

Example 18: The power transmitting device of any of examples 12 through 17, wherein the secondary processing circuitry is further configured to: independently calculate a second target power magnitude; compare the first target power magnitude to the second target power magnitude independently calculated by the second processing circuitry; responsive to determining that the first target power magnitude fails to correspond to the second target power magnitude, control the driver circuit to fail safe: wherein to fail safe comprises to terminate power transmission, and wherein to correspond to the second power comprises that the second target power magnitude is within a predetermined tolerance of the first target magnitude.

Example 19: The power transmitting device of any of examples 12 through 18, further comprising independently calculate a second target power magnitude; output the second target power magnitude to the second processing circuitry.

Example 20: The power transmitting device of example 19, wherein the second processing circuitry is further configured to: receive the second target power magnitude; compare the first target power magnitude to the second target power magnitude independently calculated by the second processing circuitry; responsive to determining that the first target power magnitude fails to correspond to the second target power magnitude, control the driver circuit to fail safe: wherein to fail safe comprises to terminate power transmission, and wherein to correspond to the second power comprises that the second target power magnitude is within a predetermined tolerance of the first target magnitude.

Example 21: The power transmitting device of any of examples 19 and 20, wherein the second processing circuitry is further configured to: receive the second target power magnitude; compare the first target power magnitude to the second target power magnitude independently calculated by the second processing circuitry; responsive to determining that the first target power magnitude fails to correspond to the second target power magnitude, control the driver circuit to output wireless electrical energy based on the lower of the first target power magnitude and the second target power magnitude.

Example 22: A method for delivering wireless power comprising sending, by first processing circuitry operatively coupled to a memory, a first target power magnitude to second processing circuitry, the second processing circuitry configured to control a driver circuit configured to drive a power transmitting antenna to transmit wireless electrical energy based on the target power magnitude, wherein the wireless electrical energy provides wireless power to an implantable medical device; comparing, by the second processing circuitry, the first target power magnitude to a threshold output power limit; and responsive to determining that the first target power magnitude is outside the threshold output power limit, controlling, by the second processing circuitry, the driver circuit to terminate power transmission.

Example 23: The method of example 22, wherein the threshold output power limit is a value on an output power profile, wherein the output power profile comprises a plurality of output power thresholds over a duration of a wireless power transfer session.

Example 24: The method of any of examples 22 and 23, further comprising calculating, by the second processing circuitry, an integral of power delivered over a predetermined duration; comparing, by the second processing circuitry, the calculated integral of power delivered to an integral dose curve stored at a memory location accessible by the second processing circuitry; and responsive to the calculated integral of power delivered exceeding the integral dose curve for the predetermined time duration, by the second processing circuitry, controlling the driver circuit to terminate power transmission.

Example 25: The method of example 24, wherein the power delivered over the predetermined duration is based on a measured power delivered by the power transmitting unit.

Example 26: The method of any of examples 24 and 25, wherein responsive to driver circuit stopping the output power for a predetermined stop duration, the second processing circuitry is configured to reset the integration calculation of power delivered.

Example 27: The method of any of examples 22 through 26, further comprising verifying, by the second processing circuitry, that, within a predetermined watchdog duration, the second processing circuitry independently calculated the target power magnitude and compared the received target power magnitude to the independently calculated target power magnitude, responsive to the watchdog duration expiring before the second processing circuitry independently calculated the target power magnitude and compared the received target power magnitude to the a threshold output power limit, outputting, by the second processing circuitry, a signal that resets the: first processing circuitry the driver circuit; and the second processing circuitry.

Example 28: The method of any of examples 22 through 27, further comprising independently calculating, by the second processing circuitry, a second target power magnitude; comparing, by the second processing circuitry, the first target power magnitude to the second target power magnitude; responsive to determining the first target power magnitude corresponds to the second target power magnitude, then controlling the driver circuit to fail safe: wherein to fail safe comprises to terminate power transmission, and wherein to correspond to the second power comprises that the second target power magnitude is within a predetermined tolerance of the first target magnitude.

Example 29: A method for delivering wireless power comprising controlling, by first processing circuitry, a wireless power transfer to an implantable medical device; receiving, by second processing circuitry, data from the implantable medical device, the data comprising information related to the wireless power transfer; estimating, by the second processing circuitry, cumulative heating for the implantable medical device based on the received data; comparing, by the second processing circuitry, the cumulative heating for the implantable medical device to a threshold; in response to the cumulative heating satisfying the threshold, selecting an action; and performing the action.

Example 30: The method of example 29, wherein the selected action includes one or more of: controlling the driver circuit to terminate power transmission, controlling the driver circuit to adjust the power transmission; stopping the output power for a predetermined stop duration; and outputting a signal that generates an alert.

Example 31: The method of any of examples 29 and 30, wherein the wireless power transfer recharges an electrical energy storage device.

Example 32: The method of any of examples 29 through 31, wherein the estimated cumulative heating comprises an estimate for CEM43.

Example 33: A non-transitory computer-readable storage medium comprising instructions that, when executed, cause processing circuitry of a computing device to: control a wireless power transfer to an implantable medical device by first processing circuitry; receive, by second processing circuitry, data from the implantable medical device, the data comprising information related to the wireless power transfer, wherein the second processing circuitry is separate from the first processing circuitry; estimate, by the second processing circuitry, cumulative heating for the implantable medical device based on the received data; compare, by the second processing circuitry, the cumulative heating for the implantable medical device to a threshold; in response to the cumulative heating satisfying the threshold, selecting an action; and performing the action.

Example 34: The non-transitory computer-readable storage medium of example 33, wherein the selected action includes one or more of: controlling the driver circuit to terminate power transmission, controlling the driver circuit to adjust the power transmission; stopping the output power for a predetermined stop duration; and outputting a signal that generates an alert.

Example 35: The non-transitory computer-readable storage medium of example 33, wherein the wireless power transfer recharges an electrical energy storage device.

Example 36: The non-transitory computer-readable storage medium of example 33, wherein the estimated cumulative heating comprises an estimate for CEM43.

In one or more examples, the functions described above may be implemented in hardware, software, firmware, or any combination thereof. For example, the various components of FIGS. 2 and 3, such as processing circuitry 30, primary processing circuitry 50, secondary processing circuitry 40, telemetry circuitry 56 and so on may be implemented in hardware, software, firmware, or any combination thereof. If implemented in software, the functions may be stored on or transmitted over, as one or more instructions or code, a computer-readable medium and executed by a hardware-based processing unit. Computer-readable media may include computer-readable storage media, which corresponds to a tangible medium such as data storage media, or communication media including any medium that facilitates transfer of a computer program from one place to another, e.g., according to a communication protocol. In this manner, computer-readable media generally may correspond to (1) tangible computer-readable storage media which is non-transitory or (2) a communication medium such as a signal or carrier wave. Data storage media may be any available media that can be accessed by one or more computers or one or more processors to retrieve instructions, code and/or data structures for implementation of the techniques described in this disclosure. A computer program product may include a computer-readable medium.

The term “non-transitory” may indicate that the storage medium is not embodied in a carrier wave or a propagated signal. In certain examples, a non-transitory storage medium may store data that can, over time, change (e.g., in RAM or cache). By way of example, and not limitation, such computer-readable storage media, may include random access memory (RAM), read only memory (ROM), programmable read only memory (PROM), erasable programmable read only memory (EPROM), electronically erasable programmable read only memory (EEPROM), flash memory, a hard disk, a compact disc ROM (CD-ROM), a floppy disk, a cassette, magnetic media, optical media, or other computer readable media. In some examples, an article of manufacture may include one or more computer-readable storage media.

Also, any connection is properly termed a computer-readable medium. For example, if instructions are transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. It should be understood, however, that computer-readable storage media and data storage media do not include connections, carrier waves, signals, or other transient media, but are instead directed to non-transient, tangible storage media. Combinations of the above should also be included within the scope of computer-readable media.

Instructions may be executed by one or more processors, such as one or more DSPs, general purpose microprocessors, ASICs, FPGAs, or other equivalent integrated or discrete logic circuitry. Accordingly, the term “processor,” as used herein, such as secondary processing circuitry 40, may refer to any of the foregoing structure or any other structure suitable for implementation of the techniques described herein. Also, the techniques could be fully implemented in one or more circuits or logic elements.

The techniques of this disclosure may be implemented in a wide variety of devices or apparatuses, including, an integrated circuit (IC) or a set of ICs (e.g., a chip set). Various components, modules, or units are described in this disclosure to emphasize functional aspects of devices configured to perform the disclosed techniques, but do not necessarily require realization by different hardware units. Rather, as described above, various units may be combined in a hardware unit or provided by a collection of interoperative hardware units, including one or more processors as described above, in conjunction with suitable software and/or firmware.

Various examples of the disclosure have been described. These and other examples are within the scope of the following claims.

What is claimed is:

1. A power transmitting device comprising:
a power transmitting antenna;
a driver circuit configured to cause the power transmitting antenna to transmit wireless electrical energy to an implantable medical device;
first processing circuitry operatively coupled to a first memory, the first processing circuitry configured to execute programming instructions stored at the first memory to determine and output a first target power magnitude; and
second processing circuitry configured to:
control the driver circuit;
receive the first target power magnitude;
compare the first target power magnitude to a threshold output power limit; and
responsive to determining that the first target power magnitude is outside the threshold output power limit, control the driver circuit to terminate power transmission.

2. The power transmitting device of claim 1, wherein the threshold output power limit is a value on an output power profile, wherein the output power profile comprises a plurality of output power thresholds over a duration of a wireless power transfer session.

3. The power transmitting device of claim 1, wherein the second processing circuitry is further configured to:
calculate an integral of power delivered over a predetermined duration;
compare the integral of power delivered to an integral dose curve stored at a memory location accessible by the second processing circuitry; and
responsive to determining that the calculated integral of power delivered exceeds the integral dose curve for the predetermined time duration, control the driver circuit to terminate power transmission.

4. The power transmitting device of claim 3, wherein the power delivered over the predetermined duration is based on a measured power delivered by the power transmitting unit.

5. The power transmitting device of claim 3, wherein responsive to driver circuit stopping the output power for a predetermined stop duration, the second processing circuitry is configured to reset the integration calculation of power delivered.

6. The power transmitting device of claim 1,
wherein the second processing circuitry is further configured to execute a watchdog function, and
wherein the watchdog function causes the second processing circuitry to verify that:
within a predetermined watchdog duration, the second processing circuitry independently calculates the target power magnitude and compares the received target power magnitude to the threshold output power limit, and
responsive to the watchdog duration expiring before the second processing circuitry independently:
calculates the target power magnitude and
compares the received target power magnitude to the independently calculated target power magnitude, the watchdog function causes the second processing circuitry to output a signal that resets the power transmitting unit.

7. The power transmitting device of claim 1, wherein the secondary processing circuitry is further configured to:
independently calculate a second target power magnitude;
compare the first target power magnitude to the second target power magnitude independently calculated by the second processing circuitry;
responsive to determining that the first target power magnitude fails to correspond to the second target power magnitude, control the driver circuit to fail safe:
wherein to fail safe comprises to terminate power transmission, and
wherein to correspond to the second power comprises that the second target power magnitude is within a predetermined tolerance of the first target magnitude.

8. The power transmitting device of claim 1, further comprising a third processing circuitry configured to:
independently calculate a second target power magnitude;
output the second target power magnitude to the second processing circuitry.

9. The power transmitting device of claim 8, wherein the second processing circuitry is further configured to:
receive the second target power magnitude;
compare the first target power magnitude to the second target power magnitude independently calculated by the second processing circuitry;
responsive to determining that the first target power magnitude fails to correspond to the second target power magnitude, control the driver circuit to fail safe:
wherein to fail safe comprises to terminate power transmission, and
wherein to correspond to the second power comprises that the second target power magnitude is within a predetermined tolerance of the first target magnitude.

10. The power transmitting device of claim 8, wherein the second processing circuitry is further configured to:
receive the second target power magnitude;
compare the first target power magnitude to the second target power magnitude independently calculated by the second processing circuitry;
responsive to determining that the first target power magnitude fails to correspond to the second target power magnitude, control the driver circuit to output wireless electrical energy based on the lower of the first target power magnitude and the second target power magnitude.

11. A method for delivering wireless power, the method comprising:
sending, by first processing circuitry operatively coupled to a memory, a first target power magnitude to second processing circuitry, the second processing circuitry configured to control a driver circuit configured to drive a power transmitting antenna to transmit wireless electrical energy based on the target power magnitude, wherein the wireless electrical energy provides wireless power to an implantable medical device;
comparing, by the second processing circuitry, the first target power magnitude to a threshold output power limit; and
responsive to determining that the first target power magnitude is outside the threshold output power limit, controlling, by the second processing circuitry, the driver circuit to terminate power transmission.

12. The method of claim 11, wherein the threshold output power limit is a value on an output power profile, wherein the output power profile comprises a plurality of output power thresholds over a duration of a wireless power transfer session.

13. The method of claim 11, further comprising:
calculating, by the second processing circuitry, an integral of power delivered over a predetermined duration;
comparing, by the second processing circuitry, the calculated integral of power delivered to an integral dose curve stored at a memory location accessible by the second processing circuitry; and
responsive to the calculated integral of power delivered exceeding the integral dose curve for the predetermined time duration, by the second processing circuitry, controlling the driver circuit to terminate power transmission.

14. The method of claim 13, wherein responsive to driver circuit stopping the output power for a predetermined stop duration, the second processing circuitry is configured to reset the integration calculation of power delivered.

15. The method of claim 11, further comprising executing, by the second processing circuitry, a watchdog function, the watchdog function comprising:
verifying, by the second processing circuitry, that, within a predetermined watchdog duration, the second processing circuitry independently calculated the target power magnitude and compared the received target power magnitude to the independently calculated target power magnitude,
responsive to the watchdog duration expiring before the second processing circuitry independently calculated the target power magnitude and compared the received target power magnitude to the threshold output power limit, outputting, by the second processing circuitry, a signal that resets the:
first processing circuitry
the driver circuit; and
the second processing circuitry.

16. The method of claim 11, further comprising:
independently calculating, by the second processing circuitry, a second target power magnitude;
comparing, by the second processing circuitry, the first target power magnitude to the second target power magnitude;
responsive to determining the first target power magnitude corresponds to the second target power magnitude, then controlling the driver circuit to fail safe:
wherein to fail safe comprises to terminate power transmission, and
wherein to correspond to the second power comprises that the second target power magnitude is within a predetermined tolerance of the first target magnitude.

17. A non-transitory computer-readable storage medium comprising instructions that, when executed, cause processing circuitry of a computing device to:
control a wireless power transfer to an implantable medical device by first processing circuitry;
receive, by second processing circuitry, data from the implantable medical device, the data comprising information related to the wireless power transfer, wherein the second processing circuitry is separate from the first processing circuitry;
estimate, by the second processing circuitry, cumulative heating for the implantable medical device based on the received data;
compare, by the second processing circuitry, the cumulative heating for the implantable medical device to a threshold;
in response to the cumulative heating satisfying the threshold, selecting an action; and
performing the action.

18. The non-transitory computer-readable storage medium of claim 17, wherein the selected action includes one or more of:
controlling the driver circuit to terminate power transmission,
controlling the driver circuit to adjust the power transmission;
stopping the output power for a predetermined stop duration; and
outputting a signal that generates an alert.

19. The non-transitory computer-readable storage medium of claim 18, wherein the wireless power transfer recharges an electrical energy storage device.

20. The non-transitory computer-readable storage medium of claim 18, wherein the estimated cumulative heating comprises an estimate for CEM43.
